(19) United States
(12) Patent Application Publication
(10) Pub. No.: US 2007/0041488 A1
(43) Pub. Date: Feb. 22, 2007

(54) Automatic balancing system and method for a tomography device

(76) Inventors: Martin Hoheisel, Erlangen (DE); Hans-Jurgen Muller, Pretzfeld (DE); Norbert Muller, Pretzfeld (DE)

Correspondence Address: SCHIFF HARDIN, LLP;PATENT DEPARTMENT, 6600 SEARS TOWER, CHICAGO, IL, 60606-6473, US

(21) Appl. No.: 10/556,101

(22) PCT Filed: Apr. 13, 2004
(86) PCT No.: PCT/EP04/03875

§ 371 Date: Aug. 28, 2006

(30) Foreign Application Priority Data
May 9, 2003 (DE) 103 20 973.5

(51) Int. Cl. H05G 1/60; A61B 6/00; G01N 23/00; G21K 1/12

(52) U.S. Cl. 378/4

(57) ABSTRACT

The invention relates to a tomography device (1), especially an X-ray computer tomography device or ultrasound tomography device, comprising a balancing device (23; 45) for reducing an imbalance (61) that was determined by means of the measuring system (2) rotating about an axis of rotation (4). The balancing device (23; 45) comprising means mounted on the measuring system (2) for variably positioning a balancing mass and a control device (25) acting upon said means and designed in such a manner that the balancing mass, controlled by the control device (25), can be positioned in a location appropriate to reduce the imbalance (61). The balancing mass can be configured as a liquid (F) that is positioned in a liquid-tight channel. The invention also relates to a balancing method according to which a mass (m) of a liquid quantity balancing the imbalance (61) is determined and a magneto- and/or electro-rheological liquid (F) is introduced into an annular channel (31; 71; 81, 83, 85) in such a quantity that for the subsequent operation a quantity of liquid (F) dependent on the determined mass (m) is present in the annular channel (31; 71 81, 83, 85).

### BACKGROUND OF THE INVENTION

1. Field of the Invention

The present invention lies in the field of imaging tomography apparatuses, in particular in the field of medical technology.

The invention is concerned with an imaging tomography apparatus, in particular an x-ray computer tomography apparatus or ultrasound tomography apparatus, with a measurement system that can rotate around a rotation axis and with a compensation device to minimize an imbalance detected in the measurement system.

The invention moreover concerns a method for minimization of an imbalance at a measurement system of a tomography apparatus, which measurement system can rotate around a rotation axis, of the type having an annular channel centered on the rotation axis and that can be filled with fluid.

2. Description of the Prior Art

In tomography apparatuses with a rapidly-rotating measurement system, existing imbalances or imbalances occurring in the course of the operation lead to a series of unwanted events. These range from unwanted noise development to excessive bearing wear and interferences in the imaging.

A computed tomography apparatus is known from DE 101 08 065 A1 that has means to determine an imbalance of the measurement system of the gantry and means for calculation of the point or those points at the measurement system of the gantry at which a weight weights should be arranged to compensate the imbalance. With such a computed tomography apparatus having such an integrated device for determination of an imbalance, it is possible to automatically check the imbalance, for example each time when the tomography apparatus is put into operation.

An automatic position correction of components of an x-ray computed tomography apparatus is disclosed in U.S. Pat. No. 5,109,397.

Devices that automatically implement a correction of the detected imbalance (known as a balancing) without a manual attachment of compensation weights being necessary are also known from the field of mechanical engineering, in particular tool engineering. For example, compensation devices are known having an annular channel centered around the rotation axis, in which annular channel a number of spheres are freely mobile. Corresponding or similar compensation devices are described in U.S. Pat. No. 3,282,127, WO 98/01733, U.S. Pat. No. 5,460,017, DE 35 09 089 A1, U.S. Pat. No. 4,075,909, EP 0 409 050 A2 and DE 44 44 992 C2.

A device for imbalance compensation with a linearly-displaceable compensation weight is described in DE 197 11 726 A1. A magnetically operable adjustment device for the compensation weight is provided that in particular comprises a magneto-rheological fluid as an adjustment means. DE 197 17 692 A1 discloses the use of electro- or magneto-rheological fluids as a coupling element.

Magneto-rheological and electro-rheological fluids are described in EP 1 219 849 A2 or in EP 0 644 253 A2. These are suspensions or emulsions of small particles in oil or in another base fluid, the particles exhibiting specific electrical or magnetic characteristics. Upon application of an electrical and/or a magnetic field, the state of the Theological fluid reversibly alters. In the field-charged state, the fluid stiffens to rigidity, meaning that its viscosity rises. Electro-viscous fluids are also disclosed in DE 41 31 142 A1 and DE 40 26 881 A1.

Purely magnetic fluids (which are also designated as ferrofluids) are to be distinguished from electro-rheological and magneto-rheological fluids. These magnetic fluids are normally a colloidal solution of small ferromagnetic particles in a base fluid. When the magnetic fluid is brought into the field of a magnet, the entire fluid is drawn towards the magnet and behaves as if the entire fluid were ferromagnetic. Such magnetic fluids are described in EP 0 644 253 A2 and in an article by Stefan Odenback, appearing in Physik in unserer Zeit, 2001, pages 122-127: “Ferrofluide—ihre Grundlagen und Anwendungen”. Ferrofluids are often used as sealing agents.

Proposals have also been made not only to use fluids as adjustment means for automatic imbalance compensation but also to use the fluid itself as a compensation mass. A corresponding balancing device according to Le Blanc mentions DE 35 09 089 A1. Arrangements of spheres in viscous fluids are also disclosed in this document. Conventional fluids as compensation mass are also disclosed in DE 3 309 387 A1, DE 102 726 A1 and DE 195 08 792 A1.

The use of heavy metal salt ions (for example mercury salt ions) movable via electrical fields for weight compensation is described in WO 01/98745. DE-PS 695 245 teaches the use of substances for imbalance compensation that can be re-liquefied by heat supply thereto.

An automatically-balancing washing machine is disclosed in the abstract of the Japanese patent application JP 03261500 A. Its compensation device has a sealed annular channel filled with a magnetic fluid. A number of electromagnets distributed around its circumference are present in the annular channel, which can be individually activated. The strength and position of the imbalance are determined by a separate imbalance determination unit. After stoppage of the washing machine, one or more electromagnets are selectively activated. The magnetic fluid thereby moves in the direction of the activated magnets and accumulates there, causing the imbalance to be reduced for the subsequent start-up of the washing machine. Due to the limited range of electromagnets of any kind, this compensation device has the disadvantage that it is only applicable given a vertical rotation axis, thus given an annular channel situated horizontally. Moreover, the strength or mass of the imbalance to be compensated is limited at the upper end, dependent the limited attractive force of the magnets and their limited capability to hold the attracted fluid in the subsequent operating state. In the subsequent rotating operating state, due to dissolution of the attracted fluid peaks the centrifugal forces produce a force countering the magnetic retention force. Moreover, for many applications the introduction of a fluid that remains in the fluid state in the rotating operating mode does not fulfill requirements for operating safety, for example with regard to residual vibrations or agitations or even leakage given a malfunction.

A compensation device for a high-speed milling cutter is known from SU 1771893 A1. In its cutting disc, this milling cutter has an annular channel (formed by a recess) that is filled with a ferromagnetic fluid. As a source for a magnetic field, a number of electromagnets aligned in the radial direction are distributed over the circumference in the sealed hollow formed in the cutting disc. The milling cutter functions as follows: in the initial state, no voltage is applied to the coils of the electromagnets, such that the ferromagnetic fluid is in the fluid state and uniformly flows or settles over the lower surface of the horizontally-situated annular channel. After the start of the rotation, the ferromagnetic fluid moves from the rotation axis of the milling cutter out to the frontal wall of the annular channel under the effect of centrifugal force. Under the influence of the imbalance, the compensation masses distribute unevenly and tend to compensate the vibrations and the imbalance. After adjustment to a stable course, a voltage is applied at the coils. A magnetic field thereby arises causing the ferromagnetic fluid should rigidly, known as “hardening”. The milling cutter is ready for operation.

The device described in SU 1771893 also has the disadvantage that only slight imbalances, for example caused by a missing tooth on the milling cutter, can be corrected. In other words: the dynamic range of the compensation device is small. The passive compensation mechanism pursued according to the SU document, in which the compensation masses should quasi-independently seek to compensate the imbalance, allows—insofar as it actually functions reliably at all—only slight imbalances to be compensated.

### SUMMARY OF THE INVENTION

An object of the present invention is based on the object to specify an imaging tomography apparatus and a method for “balancing” a tomography apparatus with which the quality of the imaging can be improved.

The above object is achieved in accordance with the invention in an imaging tomography apparatus, such as an x-ray computed tomography apparatus or an ultrasound apparatus, having a measurement system that is rotatable around a rotation axis, and that has a compensation device for reducing a detected imbalance of the measurement system, wherein the compensation device includes an annular channel in the measurement system that can be filled with a fluid, a detector that determines imbalance of the measurement system and that calculates a mass to compensate the imbalance, a reservoir, in sealed connection with the annular channel, that contains magneto-rheological fluid and/or electro-rheological fluid that is transferred into the annular channel dependent on the calculated compensating mass, and a field generator that generates a magnetic field and/or an electrical field in the annular channel to increase the viscosity of the quantity of magneto-rheological fluid and/or electro-rheological fluid therein to reduce the imbalance.

The annular channel preferably is centered on the rotation axis of the measurement system, and can be filled via an injection opening.

In such a tomography apparatus, an automatic imbalance compensation and/or an imbalance compensation without external intervention in the apparatus (for example without opening the housing) or/and an imbalance compensation without access by service personnel is possible in an advantageous manner.

The use of a fluid has the advantage that the imbalance compensation can be implemented over a large dynamic range, meaning given an imbalance that is variable over a large range.

For accommodation and guided movement of the fluid, the positioning arrangement is a fluid-sealed channel. The channel is in particular fashioned as an annular channel that can additionally be centered on the rotation axis. It can be filled, for example, via an injection opening.

The reservoir itself also can function as an imbalance compensation reservoir.

Relative to the annular channel, the reservoir is preferably mounted radially inwardly in order to minimize effects of possible imbalances in the reservoir.

In a preferred embodiment of the tomography apparatus, the reservoir is fashioned annularly, the reservoir preferably being centered on the rotation axis. Such an annular reservoir has the advantage that no imbalance is produced due to the variable content in the reservoir given a fluid exchange with the annular channel. The remaining fluid in the reservoir can be uniformly distributed over the extent of the reservoir by centrifugal force.

According to another preferred embodiment, the compensation device has a further reservoir that can likewise be connected fluid-tight with the annular channel that preferably is arranged diametrically opposite to the other reservoir and in particular at the same radial distance. This embodiment offers the advantage that the fluid necessary for compensation of the determined imbalance, which fluid is to be transferred into the annular channel, can be extracted from both reservoirs in equal parts so that no noteworthy imbalance is produced by the in both reservoirs (which reservoirs preferably are located radially inwardly anyway).

In the case of reservoirs that are not exactly diametrical opposite one another at a radially equal distances, a slight imbalance can arise due to the reservoirs, but this can be taken into account from the outset in the calculation of the required compensation mass—given known positions of both reservoirs—by a computer controlling the automatic imbalance compensation, such that the established imbalance still can be completely corrected as a result.

According to a preferred embodiment, the compensation device has at least one further annular channel that is concentric to the aforementioned annular channel and is separated from that annular channel in the direction of the rotation axis. Not only can an azimuthal imbalance of the tomography apparatus be corrected in this embodiment, but also an imbalance occurring in the axial direction

The single annular channel and/or each further annular channel each can be fashioned as an annular tube or as a (rigid or partially flexible) annular hose.

A sealing element to prevent the fluid exchange between the annular channel and the reservoir is preferably connected between the annular channel and the reservoir. The sealing element can be activated by a computer controlling the automatic imbalance compensation.

In the event that fluid must be extracted from the annular channel again for a new automatic balancing, a number of possibilities exist, of which the preferred are described in the following:

The compensation device can include a conductor piece, preceding radially outwards from the annular channel, for transport of fluid from the annular channel. This is possible due to the (virtual) rotation axis (normally horizontal), due to the vertically-standing annular channel, because the fluid automatically exits the annular channel if the outwardly preceding conductor element is positioned at the geodetically-deepest position. For example, the fluid can be guided back again into the compensation reservoir or into one of the compensation reservoirs by the outwardly preceding conductor piece, for example via a further conductor piece, in particular after the conductor element outwardly preceding and henceforth filled with fluid is brought into a position lying geodetically further above from where the fluid naturally runs back into the compensation reservoir.

Alternatively or additionally, a suction pump that can act on the annular channel for transport of fluid from the annular channel can be associated with he compensation device.

In another embodiment the compensation device has a field generator with which an electrical field and/or a magnetic field can be generated in the annular channel.

Preferred embodiments of the field generator are subsequently described:

The field in the annular channel preferably is generated with variable strengths along the annular channel.

The field generator can be formed by a number of electrodes distributed along the annular channel that can be individually charged with voltage, the electrodes preferably lying flat on the annular channel. A tomography apparatus so designed is particularly suitable for operation with an electro-rheological fluid.

The field alternatively can be formed by a number of coils distributed along the annular channel that can be individually charged with current. This variant is particularly suitable for operation with a magneto-rheological fluid.

In general, the field generator is formed by a series of field elements along the annular channel, which field elements can be activated separately.

The coils are preferably each wound around the annular channel.

To satisfy the high requirements for operating safety, in the case of a failure of the current supply that supplies the field generator or of the associated current grid, it is particularly advantageous for the field generator to be formed by a of permanent magnets distributed along the annular channel. A locally-variable charging of the annular channel with a variable magnetic field can be achieved, for example, by the permanent magnets being magnetized and/or demagnetized by the aforementioned coils.

The above object also achieved according to the invention by a method wherein
a) a mass of a fluid quantity compensating the imbalance is determined,
b) a magneto-rheological fluid and/or an electro-rheological fluid is introduced into the annular channel in a quantity that (dependent on the determined mass) is adequate for the subsequent operation of the tomography apparatus, and
c) the viscosity of the filled fluid is increased by the influence of an electrical and/or magnetic field for balanced operation of the tomography apparatus.

In contrast to known methods, in the method according to the invention a constant fluid quantity in the annular channel is not assumed. Rather, the quantity of the fluid in the annular channel is adapted via fluid exchange, dependent on the determined imbalance. Imbalances of a few grams up to many kilograms can thereby be compensated.

As used herein fluid exchange encompasses both a fluid feed and a fluid transport into or out of the annular channel

The tomography apparatus with the compensation device described above is in particular suitable for implementation of the method according to the invention. Advantages and embodiments mentioned with regard to the tomography apparatus analogously apply for the method.

In connection with the invention, any closed or sealable fluid volume that essentially proceeds in the circumferential direction around the rotation axis, and thus is annular as seen in a direction parallel to the rotation axis is substantially understood as an annular channel. The annular channel can be fashioned as an annular recess or as a separately introduced annular tube, for example as an annular hose or as an annular tube of round or angled cross section.

According to a preferred embodiment, the magneto-rheological or electro-rheological fluid contains particles that can be polarized in an electrical and/or in a magnetic field. Such a rheological fluid can be stabilized particularly well for the rotational operation of the tomography apparatus and moreover can be held, with particularly definition and specification, at a specific circumferential position.

According to a preferred development of the method, the steps a) through c) are repeated during the operation time, the lifespan, the service life or the availability time of the tomography apparatus in order to compensate for an imbalance that has changed in the interim.

The method according to the invention is suited both for an operating mode in which the fluid automatically moves into an azimuthal position necessary for compensation of the imbalance and for an operating mode in which the fluid is actively positioned at a previously determined position necessary for compensation of the imbalance.

With regard to the first-cited mode, the tomography apparatus is preferably moved into such a fast rotation, in particular with a rotation frequency above the resonance frequency that the fluid introduced into the annular channel naturally moves to an azimuthal position necessary for compensation of the imbalance. In this operating mode, the electrical and/or magnetic field can be uniformly activated over the entire circumference of the annular channel via corresponding field means. A selective local activation of the field occurring at a specific, azimuthal position is not necessary.

With regard to the second cited mode, according to a preferred embodiment position of the fluid quantity compensating the imbalance is also determined (in addition to the mass) in the method according to the invention, and the fluid introduced into the annular channel is positioned (meaning in particular held) in the annular channel using the determined position in the azimuthal direction.

Three variants are subsequently described according to which the introduced fluid is preferably brought into the desired azimuthal position:

i) Due to the normally horizontal rotation axis of the measurement system of the tomography apparatus, the introduced fluid is brought to the determined position, in that the measurement system is positioned such that it comes to lie at the geodetically-lowest point with the determined position, such that the introduced fluid collects there. The annular channel is thus only partially filled with fluid, and preferably only one fluid quantity, corresponding to a previously determined mass necessary for the compensation (step a) of the above method), is injected.

In this variant, the activation of the field for the subsequent rotation operation of the tomography apparatus then occurs locally at the determined position or uniformly distributed over the entire circumference.

The local activation of the field has the advantage that a further degree of freedom in the precise positioning and fixing of the introduced fluid in the annular channel is achieved via an additional parameter. Precise results are therewith already possible with slight calculational effort. Moreover, in particular in the case of the field activation uniformly activated over the circumference, the shape of the fluid pool accumulating at the geodetically-lowest point in the calculation of the fluid mass can be taken into account, such that a local field activation can be omitted for many application fields.

ii) The tomography apparatus also can be shifted into rotation such that the introduced fluid is distributed along the annular channel as a consequence of the centrifugal force. The introduced fluid is then locally hardened at the determined position due to the action of the electrical or magnetic field, with the field acting on the fluid with a strength equivalent to the determined mass and/or with an effective volume equivalent to the determined mass.

For local hardening, a number of field element distributed along the circumference of the annular channel are present. For example, the effective volume can be influenced by the number of the activated field elements.

iii) A distribution along the annular channel can also be effected by initially filling the entire volume of the annular channel with fluid to the greatest possible extent, in particular under pressure. The local hardening then occurs as under ii).

According to another preferred development, after local hardening of the fluid and before the subsequent operation of the tomography apparatus, a remaining, non-hardened portion of the fluid is removed from the annular channel. An overabundance of fluid can thus initially be filled into the annular channel. The removal of the superfluous fluid quantity can also occur successively, whereby in each step the field strength or the effective volume are varied until an optimal balancing result is achieved.

To allow space for a possible reservoir from which the fluid is extracted for the annular channel, it is advantageous to only partially fill the annular channel at the beginning of the balancing event or to fill the annular channel dependent on a mass that was previously determined as necessary for the compensation (step a) of the above method).

### DESCRIPTION OF THE DRAWINGS

FIG. 1 schematically illustrates a first exemplary embodiment of a tomography apparatus constructed and operating in accordance with the principles of the present invention.

FIG. 2 schematically illustrates a second exemplary embodiment of a tomography apparatus constructed and operating in accordance with the principles of the present invention.

FIG. 3 schematically illustrates a third exemplary embodiment of a tomography apparatus constructed and operating in accordance with the principles of the present invention, in a first operating state.

FIG. 4 schematically illustrates the tomography apparatus of FIG. 3, in a different operating state.

FIG. 5 schematically illustrates the tomography apparatus of FIGS. 2 and 3 in a further operating state.

FIG. 6 schematically illustrates a fourth exemplary embodiment of a tomography apparatus constructed and operating in accordance with the principles of the present invention.

FIG. 7 schematically illustrates a fifth exemplary embodiment of a tomography apparatus constructed and operating in accordance with the principles of the present invention.

FIG. 8 schematically illustrates a sixth exemplary embodiment of a tomography apparatus constructed and operating in accordance with the principles of the present invention.

FIG. 9 illustrates an electrical field generator for use in those embodiments, among the above embodiments, that make use of an electro-rheological fluid.

FIG. 10 shows a magnetic field generator for use in those embodiments, among the above exemplary embodiments, which make use of a magneto-rheological fluid.

### DESCRIPTION OF THE PREFERRED EMBODIMENTS

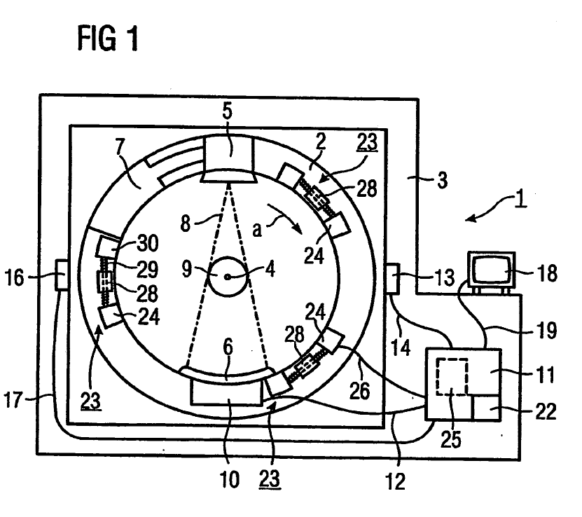

FIG. 1 shows an x-ray computed tomography apparatus 1, as an example of a rotatable device. The tomography apparatus 1 has a measurement system 2 as a rotatable part of the gantry, The measurement system 2 is capable of rotating in a stationary housing 3 around a virtual horizontal rotation axis 4 perpendicular to the drawing plane. A number of components are arranged on the measurement system 2, namely an x-ray source 5, an x-ray radiation detector 6 opposite the x-ray source 5 and a cooling device 7 (only schematically indicated) for dissipation of heat that is generated by an x-ray tube of the x-ray source 5 in the operation of the computer tomography apparatus 1. In the operation of the computer tomography apparatus 1, the measurement system 2 rotates around the rotation axis 4, whereby a fan-shaped x-ray beam 8 emanating from the x-ray source 5 penetrates a measurement field 9 at various projection angles and strikes the radiation detector 6. The resulting output signals of the radiation detector 6 are supplied to a data processing device 10 that forms measurement values that are supplied to a control and image processing computer 11 of the computer tomograph 1. From these values, the control and image processing computer 11 calculates an image of a patient (not shown) located in the measurement field 9. The data processing device 10 is connected with the control and image processing computer 11 via a data path that, for example, includes (in a manner not shown) a slip ring system or a wireless optical transmission path. The electrical connections of the x-ray source 5 and of the radiation detector 6 can also be effected in a known manner via slip rings.

In order to be able to reconstruct images from the measurement values, a position sensor 13 is arranged on the housing 3 of the computer tomograph 1. The position sensor 13, in the operation of the measurement system 2, continuously detects the position of this rotating part 2 relative to the housing 3 and transmits this information to the control and image processing computer 11 by via a line 14.

Imbalances in the measurement system 2, both radially and axially relative to the rotation axis 2, normally arise in the manufacturing of the computer tomograph 1, such that the measurement system 2 does not rotate exactly relative to its rotation axis 4. Such imbalances also arise in the course of the operation of the computer tomograph 1, for example due to changes of the coolant in the cooling device 7 or due to tolerance build-up or exchange of electronic or other components on the rotatable measurement system 2. Such imbalances are unwanted since they lead to blurred images produced with the computer tomograph 1 or even to damage of the mechanical mounting.

To determine the imbalance and for calculation of a mass to compensate the imbalance and optionally of a position of this mass, the tomography apparatus 1 has a number of measurement sensors 16 fashioned as vibration of acceleration sensors, which measurement sensors are connected with the control and image processing computer 11 via lines 17. During the rotation of the measurement system 2, one of the measurement sensors 16 detects resulting vibrations in the radial direction, in contrast to which a different measurement sensor 16 detects the vibrations resulting in the axial direction during the rotation of the measurement system 2.

A monitor 18 on which the result of an imbalance determination can be displayed is associated via a line 19 with the control and image processing computer 11 in which a balancing software is installed. A memory 22 is present for storage of such a result.

The control and image processing computer 11 automatically determines the imbalance of the measurement system 2 each time the computer tomography 1 is brought online.

Details of the determination of an imbalance and the calculation of a mass compensating the imbalance, can be found in DE 101 08 065 A1, the disclosure content is incorporated herein by reference.

In the first exemplary embodiment shown in FIG. 1, a compensation device 23 is present for dynamic compensation or for variable correction of an imbalance (not explicitly shown in FIG. 1), the compensation device 23 being composed of electrically activatable motors or adjustment elements 24 at a number (here: three) of positions that differ azimuthally and that are not diametrically complementary. A rigid, heavy and metallic compensation body 28 can be moved in the tangential direction by means of the adjustment elements 24 that are connected with a control device 25 via data connections 26. The control device 25 (formed as a functional module in the control computer 11) positions the compensation body 28 (in the event that this is necessary) at a different point required for the imbalance compensation and previously calculated by means of known balancing software. Each compensation body 28 can be moved by means of a threaded rod 29 that can be driven by the appertaining adjustment element 24 and is supported in a rotatable fashion in a counter-bearing 30 that is azimuthally spaced from the associated adjustment element 24.

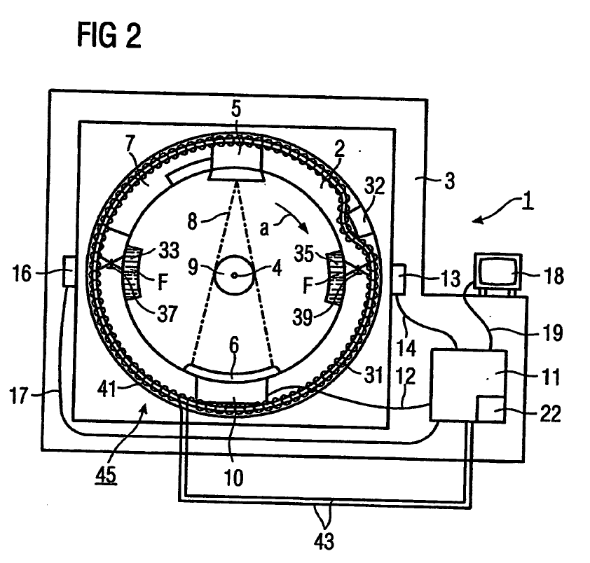

In the second exemplary embodiment shown in FIG. 2, an annular channel 31 fashioned as a flexible hose is mounted along the circumference of the measurement system 2 for dynamic compensation or for variable correction of the imbalance (not explicitly shown in FIG. 2). Due to the flexibility of the annular hose, it is possible to place this around an exemplarily indicated component 32. This is particularly advantageous in the shown computer tomography apparatus 1 because a number of electrical and mechanical components must be arranged on the measurement system (gantry) 2.

Two reservoirs 33, 34 filled with an electro-rheological or magneto-rheological fluid F are also mounted on the rotatable measurement system 2. These lie symmetrically and at an equal interval opposite one another relative to the rotation axis 4. The reservoirs 33, 35 lie radially further inwards relative to the annular channel 31, such that a possible imbalance produced by the reservoirs 33, 35 is kept low from the outset in an advantageous manner. Given an exactly symmetrical execution of both reservoirs 33, 35 and given their symmetrical operation, a mounting location is however also possible that lies radially further outwards relative to the annular channel 31.

The reservoirs 33, 35 are connected via sealing elements 37, 39 (which can be operated by the control and image processing computer 11 with regard to opening and closing) with the annular channel 31, such that a fluid transfer—for example driven by gravity or by centrifugal force) can occur between the reservoirs 33, 34 and the annular channel 31. The annular channel 31 can be internally charged with a magnetic field by a field generator 41 designed as an annular coil, such that a magneto-rheological fluid F injected into the annular channel 31 can be hardened. The annular coil is continuously wound around the annular channel 31 along its entire extent and is connected with the control and image processing computer 11 via a line 43. A magnetic field homogeneous to the greatest possible extent can be generated in this manner in the annular channel 31 along its circumference.

The annular channel 31, the reservoirs 33, 35 with their sealing elements 37, 39 and the field generator 41 in combination form a compensation device 45 for reduction of the aforementioned imbalance. To reduce the imbalance, a mass m of a fluid quantity that compensates the imbalance is initially determined by means of the measurement sensor 16 and the corresponding quantity of a magneto-rheological fluid F is introduced into annular channel 31 in equal parts from the reservoirs 33, 35. The measurement system 2 of the tomography apparatus 1 is then shifted into fast rotation. The rotation frequency is at least increased up to the resonance frequency that was previously determined (for example by means of the measurement sensor 16) during a calibration. From the resonance frequency, the angular position of the compensation mass introduced as a fluid F changes by 180° relative to the imbalance and the compensation mass automatically migrates to an azimuthal position necessary for compensation of the imbalance, which azimuthal position lies precisely diametrically opposite a determined imbalance mass that is idealized as a point. After this process has concluded, the fluid F is exposed to a magnetic field by charging the field generator 41 with electrical current. The fluid thereby changes into a gelatinous, more solid medium (“hardens”) and stably remains at the required position.

The tomography apparatus 1 is now in a balanced state and ready for operation.

The electro-rheological or magneto-rheological fluid used for reduction of the imbalance is formed by a base fluid in which are distributed particles that can polarize in an electrical and/or in a magnetic field. The fluid is in particular fashioned as a (preferably non-colloidal) suspension. Such polarizable, rheological fluids have the advantage that, in the presence of a magnet, they are not attracted or are barely attracted to this. The possibility for a precise imbalance compensation with high dynamic thereby results in an advantageous manner. The fluid preferably exhibits no ferromagnetic properties. The particles (whose dipole moment, for example, only exists under the influence of the field) preferably exhibit a size in the range greater than 0.5 μm, in particular in the range from 0.1 μm to 10 μm. They are in particular predominantly composed of iron, for example soft iron, steel, cobalt or carbonyl iron. The base fluid is preferably predominantly composed of water and/or an oil, in particular a synthetic or silicon-based oil.

Due to the use of interference-free permanent magnets, in comparison with electro-rheological fluids the use of magneto-rheological fluids is particularly advantageous for practical operation. The higher density of the magneto-rheological fluids, which improves the dynamic range and the required design space for the compensation device, is also advantageous.

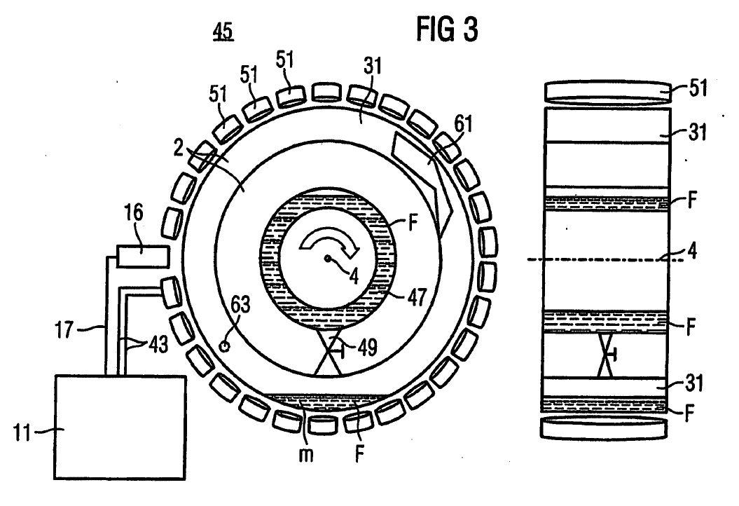

FIG. 3 shows a third exemplary embodiment of a tomography apparatus according to the invention in which, for reasons of better presentation capability, essentially only the compensation device 45 is still shown. In this embodiment, an annular reservoir 47 is present instead of two reservoirs, which annular reservoir 47 is mounted concentrically on the rotation axis 4 and radially further inwards relative to the annular channel 31 fashioned as an annular tube and exhibits a smaller diameter than the annular channel 31. The reservoir 47 is connected with the radially-symmetrical annular channel 31 via a control valve or sealing element 49 functioning in the same manner as the sealing elements according to FIG. 1.

A number of separately activatable field elements 51 are distributed along the circumference of the annular channel 31 as a field means 41 for charging of the inside of the annular channel 31 with an electrical and/or a magnetic field. It is thereby possible to generate the field in the annular channel 31 with variable strengths along its curve. The field elements 51 are fashioned as electromagnets or as capacitors and can be switched individually or in groups.

For compensation of a schematically indicated, idealized imbalance 61, a mass m of a fluid quantity compensating the imbalance 61 as well as the position 63 of this fluid quantity are initially determined by means of the measurement sensor 16 and the computer 11 evaluating its data. The reservoir 47 with its sealing element 49 is subsequently positioned in the geodetically lowest-lying point, such that the fluid F automatically flows from the reservoir 47 into the annular channel 31 after an opening of the sealing element 49. Using a time control of the sealing element 49, it is thereby ensured that the injected fluid quantity corresponds to the previously-determined mass m. The annular channel 31 is only partially filled. To support the fluid injection, a pump (not shown) can be present that is controlled by the computer 11.

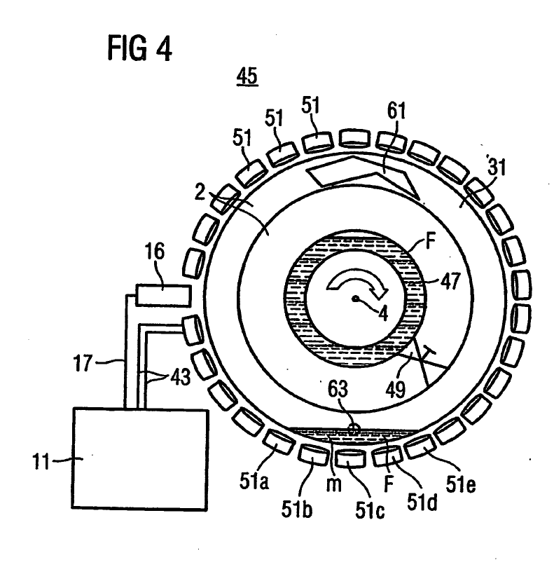

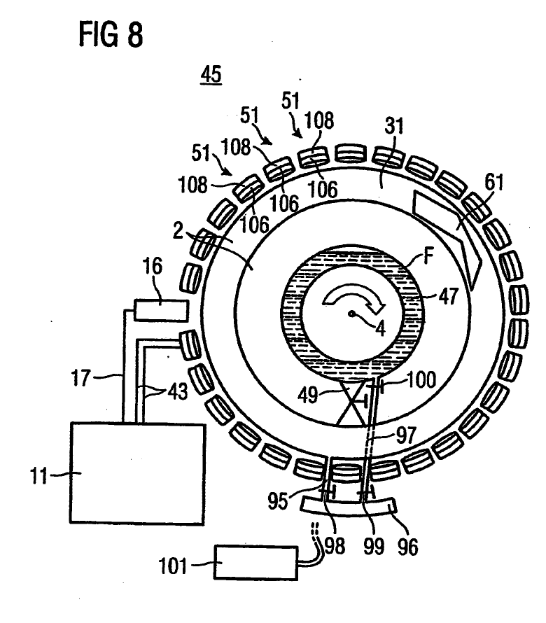

As a next step, as shown in FIG. 4 the measurement system 2 of the tomography apparatus 1 is positioned such that the fluid F introduced into the annular channel 31 automatically flows into the determined position 63. This occurs by the determined position 63 being brought to the lowest position (6 o'clock position). In this state, the fluid F located in the annular channel 31 is now charged with an electrical or magnetic field by means of the field means 41. It is sufficient to activate those field elements 51a, 51b, 51c, 51d, 51e that can act on the fluid F in the annular channel 31. The fluid F hardens at the desired point due to the field effect. Via the precise number of the activated field elements, it is possible, as an additional degree of freedom, to even precisely tune the quantity of the compensating fluid. For example, after a test pass the control software could decide to deactivate the edge-side elements 51a, 51e, such that a remaining, non-hardened portion of the fluid F can be removed from the annular channel 31 before the subsequently operation of the tomography apparatus 1. For example, the procedures described in connection with FIG. 8 can be used for this purpose.

After the field elements 51a through 51e have been activated in the state described in FIG. 4, the tomography apparatus 1 is ready for operation.

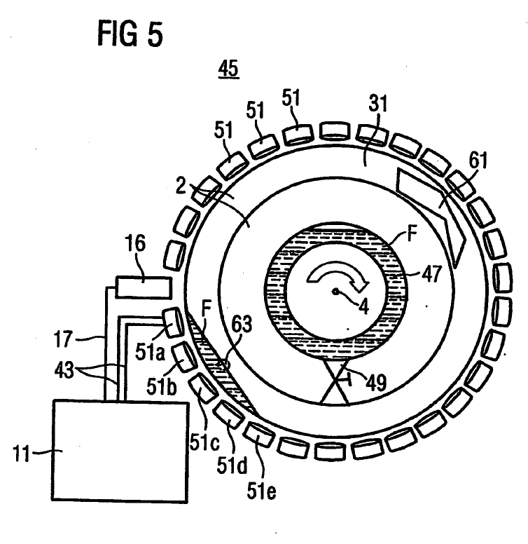

In the subsequent operation of the tomography apparatus 1, as shown in FIG. 5, the field elements 51a through 51e remain activated and the measurement system 2 is shifted into fast rotation. The introduced fluid F always remains at the previously determined position 63, thus diametrically opposite the imbalance 61. The tomography apparatus 1 is balanced.

As an alternative to the procedure described in the preceding, in which the fluid F was essentially azimuthally positioned at the =b 6 o'clock position, a procedure supported by centrifugal force is also possible: after the fluid F has been introduced into the annular channel 31 in large quantity and this is, for example, entirely or almost entirely filled, the measurement system 2 is placed into rotation such that the introduced fluid F uniformly distributes along the annular channel 31 as a consequence of the centrifugal force. Since, as described in the first procedure, the mass m necessary for compensation but also as well as the position 63 of this mass m at which the fluid is to be hardened and has to remain in the subsequent continuous operation have been determined beforehand, the field elements 51a through 51e located at this position 63 can now be selectively activated. The fluid F distributed over the circumference of the annular channel 31 is then only hardened in a specific sector. The control computer 11 can determine how many of the field elements 51a through 51e must be activated in order to achieve a specific effective volume of the field and thus to harden the desired, previously determined mass m of the fluid F. Alternatively or additionally, the strength of the activation of the individual field elements can also be drawn upon for selection of the desired fluid quantity m.

After such local hardening of the fluid F, the non-hardened portion of the fluid F remaining at the non-activated circumferential points of the annular channel 31 is removed from the annular channel 31 before the subsequent operation of the tomography apparatus 1. The tomography apparatus 1 is balanced and ready for operation.

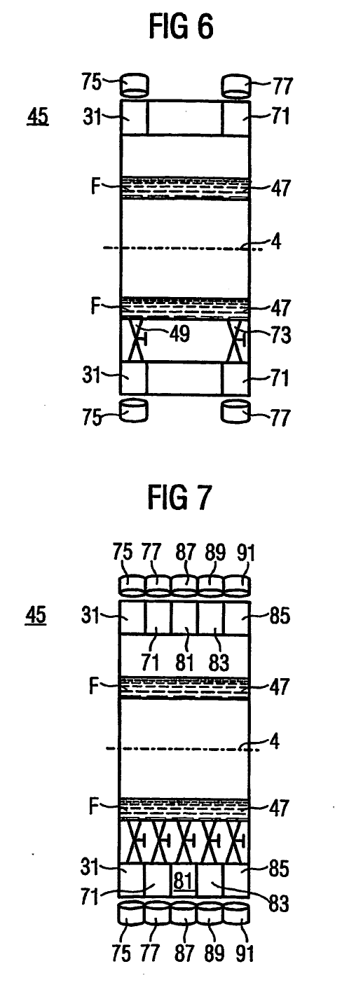

In the fourth exemplary embodiment (representation only in the slice plane parallel to the rotation plane) shown in FIG. 6 of a tomography apparatus 1 according to the invention, in addition to the first annular channel 31 a further annular channel 71 of the same diameter is present that is arranged concentric to the first annular channel 31 and separated from this in the direction of the rotation axis 4. In this example, as also in the example according to FIGS. 2 through 4, the reservoir 47 is fashioned as a hollow cylinder ring. It is connected with the respective annular channels 31, 71 via separate sealing elements 49, 73. The arrangement of a number of annular channels 31, 71 has the advantage that—in addition to an azimuthal imbalance—an axial imbalance occurring in the direction of the rotation axis 4 can also be compensated. The requirement for this is that the measurement sensor 16 (see FIG. 1) is fashioned for determination of imbalances in both directions, for example via two separate sensors.

A series of field elements 75 and a series of field elements 77 are respectively distributed along the circumference of the annular channels 31 and 71 (see FIGS. 2 through 4).

Moreover, the exemplary embodiment according to FIG. 6 is largely identical with the exemplary embodiment according to FIGS. 3 through 5.

A modification of the exemplary embodiment according to FIG. 5 is shown with a fifth exemplary embodiment in FIG. 7. In this exemplary embodiment, overall five annular channels 31, 71, 81, 83, 85 are arranged next to one another without gaps in the direction of the rotation axis 4. Each of the annular channels 31, 71, 81, 83, 85 is connected with the annular compensation reservoir 47 via a separate, separately-activatable sealing element. Moreover, a separate series of field elements 75, 77, 87, 89, 91 is associated with each annular channel 31, 71, 81, 83, 85. A particularly fine balancing is possible with the compensation device 45 according to FIG. 6.

In FIGS. 6 and 7, essentially only the compensation device 45 of the tomography apparatus 1 is respectively shown.

In the sixth exemplary embodiment shown in FIG. 8, which is in large part identical with the exemplary embodiment according to FIGS. 3 through 6, two alternative or parallel possibilities for removal of excessive fluid F in the annular channel 31 are initially shown:
a) A guide element 95 leads radially outwards from the annular channel 31 into a discharge reservoir 96. In the shown position of the measurement system 2, a fluid F located in the annular channel 31 would thus flow into the guide element 95 and the discharge reservoir 96 under the influence of gravity. After this has occurred, the measurement system 2 is rotated by 180°, such that the discharge reservoir 96 comes to lie at the 12 o'clock position. In this position, the fluid located in the discharge reservoir 96 automatically flows through a conductor connection 97 back into the reservoir 47 under the influence of gravity. In order to ensure this operating mode, valves 98, 99, 100 that can be activated by the computer 11 are present.
b) A vacuum pump or suction pump 101 (associated with the tomography apparatus 1) with whose help the excessive fluid F can be removed from the annular channel 31 can connect or be connected to the annular channel 31.

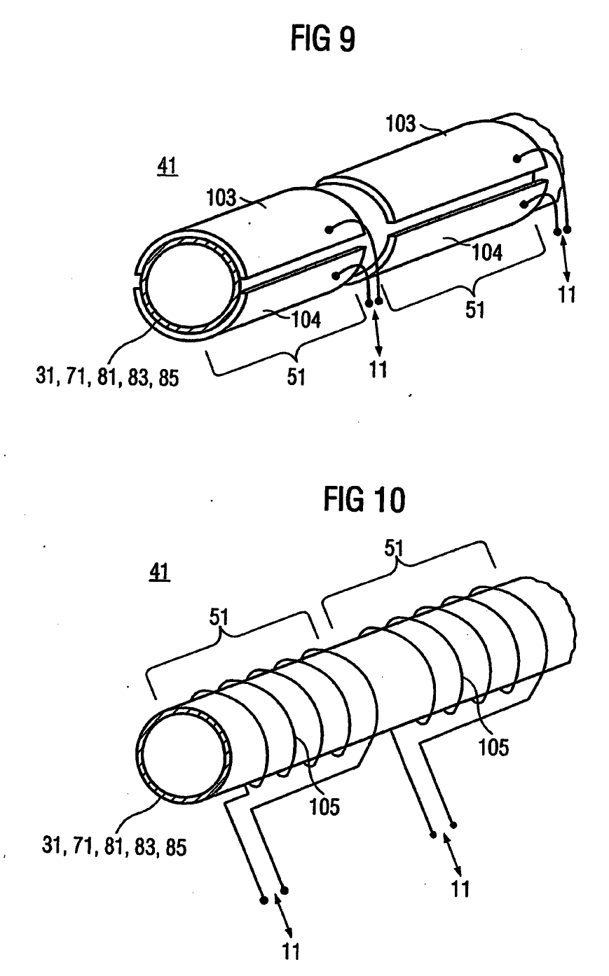

In FIGS. 9 and 10, possible embodiments of the field elements 51 are reproduced as they are shown in FIGS. 3 through 8.

According to FIG. 9, each of the field elements 51 serving as a field generator 41 and strung along the annular channel 31 (shown as an example in FIG. 9, but also valid for channels 71, 81, 83, 85) is composed of two electrodes 103, 104 that can be individually charged with electrical voltage. The electrodes 103, 104, which are adapted to the shape of the outer contour of the annular hose or annular tube, are fashioned optimally large in area and optimally, comprehensively covering the outer surface of the annular tube or annular hose. The charging of the elements 103, 104 with electrical voltage occurs controlled by the computer 11.

According to FIG. 10, the field elements 51 are fashioned for charging of the fluid F with a magnetic field. Each of the elements 51 has a coil 105 with a number of windings, the coil 105 being wound around the annular hose or the annular tube. The charging of each coil 105 is controlled by the computer 11.

In order to prevent a re-liquefaction of the fluid F introduced into the annular channel 31 (shown as an example in FIG. 10, but also valid for channels 71, 81, 83, 85) given failure of the current grid or the current feed or given interruption of the current-supplying lines, it is advantageous to design each of the field elements 51 as a separate permanent magnet 106 and a separate coil 108 acting on it. This variant is indicated in FIG. 8. Each of the coils 108 is fashioned such that the associated permanent magnet 106 thereof can be magnetized and demagnetized.

Although modifications and changes may be suggested by those skilled in the art, it is the invention of the inventors to embody within the patent warranted heron all changes and modifications as reasonably and properly come within the scope of their contribution to the art.

We claim as our invention:

1-33. (canceled)

34. An imaging tomography apparatus comprising:
a measurement system rotatable around a rotation axis, said measurement system being subject to imbalance during rotation thereof; and
a compensation device that mechanically interacts with said measurement system to compensate said imbalance, said compensation device comprising an annular channel in said measurement system;
an imbalance detector that detects said imbalance in said measurement system and determines a compensation mask to compensate said imbalance;
a reservoir containing at least one rheological fluid selected from the group consisting of magneto-rheological fluids and electro-rheological fluids;
a fluid transfer arrangement connecting said reservoir to said annular channel and being operable to transfer a quantity of said rheological fluid from said reservoir into said annular channel dependent on said compensating mass; and
a field generator disposed to generate at least one field, selected from the group consisting of magnetic fields and electrical fields, in said annular channel to interact with rheological fluid transferred into the annular channel from the reservoir to increase the viscosity thereof to compensate said imbalance.

35. An imaging tomography apparatus as claimed in claim 34 wherein said annular channel is centered in said measurement system relative to said rotation axis.

36. An imaging tomography apparatus as claimed in claim 34 wherein said reservoir is disposed radially further inwardly relative to said rotation axis, than said annular channel.

37. An imaging tomography apparatus as claimed in claim 34 wherein said reservoir is an annular reservoir and is centered on said rotation axis.

38. An imaging tomography apparatus as claimed in claim 34 wherein said reservoir is a first reservoir and wherein said fluid transfer device is a first fluid transfer device, and wherein said compensation device comprises a second reservoir, also containing said rheological fluid, and a second fluid transfer device connecting said second reservoir to said annular channel and operable to transfer said rheological fluid from said second reservoir into said annular channel, in combination with transfer of said rheological fluid into said annular channel from said first reservoir, dependent on said compensation mass, said second reservoir being disposed opposite said first reservoir.

39. An imaging tomography apparatus as claimed in claim 34 wherein said annular channel is a first annular channel, and wherein said compensation device comprises at least one further annular channel disposed concentrically in said measurement system relative to said first annular channel, and separated from said first annular channel in a direction along said rotation axis.

40. An imaging tomography apparatus as claimed in claim 34 wherein said annular channel is an annular conduit within said measurement system.

41. An imaging tomography apparatus as claimed in claim 34 wherein said annular channel is an annular hose carried by said measurement system.

42. An imaging tomography apparatus as claimed in claim 34 wherein said fluid transfer element comprises a selectively openable sealing element that prevents re-transfer of said rheological fluid from said annular channel back into said reservoir.

43. An imaging tomography apparatus as claimed in claim 34 wherein said fluid transfer element comprises a guide element proceeding radially outwardly from said annular channel allowing transfer of fluid from said annular channel.

44. An imaging tomography apparatus as claimed in claim 43 wherein said compensation device comprises a suction pump acting on said annular channel to cause said fluid to be transferred from said annular channel.

45. An imaging tomography apparatus as claimed in claim 34 wherein said field generator is operable to generate said at least one field with a variable strength along said annular channel.

46. An imaging tomography apparatus as claimed in claim 34 wherein said rheological fluid is an electro-rheological fluid, and wherein said field generator comprises a plurality of electrodes distributed along said annular channel, and a power source connected to each of said electrodes and operable to individually charge said anodes with voltage.

47. An imaging tomography apparatus as claimed in claim 34 wherein said rheological fluid is a magneto-rheological fluid, and wherein said field generator comprises a plurality of coils distributed along said annular channel, and a current source operable to individually charge said coils with current.

48. An imaging tomography apparatus as claimed in claim 47 wherein said coils are wound around said annular channel.

49. An imaging tomography apparatus as claimed in claim 34 wherein said rheological fluid is a magneto-rheological fluid and wherein said field generator comprises a plurality of permanent magnets distributed along said annular channel.

50. An imaging tomography apparatus as claimed in claim 49 wherein said field generator further comprises a plurality of coils distributed along said annular channel, and an operating unit connected to said coils to selectively, individually operate said coils to respectively magnetize and demagnetize said permanent magnets.

51. An imaging tomography apparatus as claimed in claim 34 wherein said measurement system is an x-ray computed tomography measurement system.

52. An imaging tomography apparatus as claimed in claim 34 wherein said measurement system is an ultrasound measurement system.

53. A method for reducing an imbalance of a measurement system of a tomography apparatus, said measurement system being rotatable around a rotation axis, and said measurement system having an annular channel centered on said rotation axis, said method comprising the steps of:
determining a mass of a fluid quantity for compensating said imbalance;
storing a rheological fluid, selected from the group consisting of magneto-rheological fluids and electro-rheological fluids, in a reservoir and transferring a selected quantity of said rheological fluid from said reservoir into said annular channel dependent on said mass; and
generating at least one field, selected from the group consisting of magnetic fields and electrical fields, that interact with said rheological fluid in said annular channel to increase the viscosity thereof during rotation of said measurement system to compensate said imbalance.

54. A method as claimed in claim 53 comprising employing a fluid, as said rheological fluid that contains particles that can be polarized in said at least one field.

55. A method as claimed in claim 53 comprising repeating the steps of determining said mass, transferring said rheological fluid from said reservoir into said annular channel and increasing the viscosity of the fluid in the annular channel at selected times to differently compensate for different imbalances of said measurement system occurring over time.

56. A method as claimed in claim 53 wherein said measurement system has a resonance frequency associated with rotation thereof, and rotating said measurement system at a fast rotational speed, exceeding said resonance frequency to cause said rheological fluid transferred into the annular channel to automatically move to an azimuthal position for compensating said imbalance.

57. A method as claimed in claim 53 comprising, in addition to said mass, determining a position of a quantity of said rheological fluid, dependent on said mass, to compensate said imbalance, and positioning said rheological fluid introduced into the annular channel from the reservoir at a position in the azimuthal direction of said annular channel dependent on said determined position.

58. A method as claimed in claim 57 comprising positioning said rheological fluid in said annular channel by rotating said measurement system to cause a geodetically-lowest point of said measurement system to occupy said position, to cause said Theological fluid in said annular channel to collect at said geodetically-lowest point.

59. A method as claimed in claim 57 comprising distributing said rheological fluid in said annular channel by centrifugal force during rotation of said measurement system by completing filling said annular channel with said rheological fluid, and locally hardening said rheological fluid in said annular channel at said position by selected operation of said field generator.

60. A method as claimed in claim 59 comprising removing any non-hardened portion of said rheological fluid from said annular channel after said locally hardening of said rheological fluid, before rotating said measurement system.
